(19) United States
(12) Patent Application Publication
(10) Pub. No.: US 2025/0261866 A1
(43) Pub. Date: Aug. 21, 2025

(54) BLOOD PRESSURE DETECTION APPARATUS BASED ON ARTERIOLAR PHOTOPLETHYSMOGRAM

(71) Applicant: HANGZHOU MEGASENS TECHNOLOGY CO., LTD., Hangzhou (CN)

(72) Inventors: Congcong Zhou, Hangzhou (CN); Jianjun Wang, Hangzhou (CN)

(21) Appl. No.: 18/842,077

(22) PCT Filed: May 16, 2023
(86) PCT No.: PCT/CN2023/094497

§ 371 Date: Apr. 17, 2025

(30) Foreign Application Priority Data
May 26, 2022 (CN) 202210584491.3

(51) Int. Cl. A61B 5/021; A61B 5/00; A61B 5/01; A61B 5/0205; A61B 5/022

(52) U.S. Cl. CPC A61B 5/02108; A61B 5/01; A61B 5/02055; A61B 5/02241; A61B 5/6826; A61B 5/6838; A61B 5/6885; A61B 5/7267; A61B 2560/0462; A61B 2562/0219; A61B 2562/0247; A61B 2562/0271; A61B 2562/046

(57) ABSTRACT

A blood pressure (BP) detection apparatus and method based on an arteriolar photoplethysmogram (APPG) is disclosed. The BP detection apparatus includes a ring structure, a photoplethysmogram (PPG) detection unit, a temperature sensing unit, a pressure sensing unit and a microcontroller unit (MCU) provided at a bottom of the ring structure, and spring control units respectively provided at two inner sides of the ring structure. Under an adjustment amount of the spring control unit determined according to a pressure, the MCU calculates an APPG according to a PPG detected by the PPG detection unit, and a temperature detected by the temperature sensing unit, and detects a BP according to the APPG. The BP detection apparatus has a simple structure and a simple operation, and makes an obtained signal achieve a high signal-to-noise ratio (SNR) and a high accuracy.

### CROSS REFERENCE TO RELATED APPLICATION

This application is a national stage application of International Patent Application No. PCT/CN2023/094497, filed on May 16, 2023, which claims priority to Chinese Patent Application No. 202210584491.3, filed with the China National Intellectual Property Administration (CNIPA) on May 26, 2022, and entitled “BLOOD PRESSURE DETECTION APPARATUS BASED ON ARTERIOLAR PHOTOPLETHYSMOGRAM”, which is incorporated herein by reference in its entirety.

### TECHNICAL FIELD

The present disclosure relates to the technical field of blood pressure (BP) detection, and in particular to a BP detection apparatus based on an arteriolar photoplethysmogram (APPG).

### BACKGROUND

As an important physiological parameter of the human body, the blood pressure (BP) can reflect a cardiovascular functional state. Hypertension makes the human body riskier to multiple cardiovascular diseases (CVDs) such as cerebral strokes, coronary artery diseases, cardiac failures, atrial fibrillation (AF) and peripheral vascular diseases. Monitoring change trends of the BP in different cardiovascular states is helpful to better understand pathogenesis of the CVDs and take effective measures.

When the BP is predicted with a photoplethysmogram (PPG) signal, according to a physiological structure of the human body, the relatively stable PPG signal is usually obtained by measuring vessel-rich parts, specifically an earlobe or a wrist at present. However, the signal on these parts has a poor quality and a low signal-to-noise ratio (SNR).

Besides, when the BP is predicted with the PPG signal, the PPG signal is directly measured in the conventional art. Due to different penetration depths of different wavelengths, direct measurement of the PPG signal involves many volume fluctuations in capillaries, arterioles, tissue fluids or even veins, thus causing large signal interferences and inaccuracy of the PPG signal. Therefore, when the BP is predicted, other PPG interfering signals need to be removed. In contrast, the PPG at the arterioles is the most direct and most accurate to predict the BP.

### SUMMARY

An objective of the present disclosure is to provide a BP detection apparatus based on an APPG, to improve an accuracy of BP prediction.

To achieve the above objective, the present disclosure provides the following technical solutions:
A BP detection apparatus based on an APPG includes a ring structure, a PPG detection unit, a temperature sensing unit, a pressure sensing unit and a microcontroller unit (MCU) provided at a bottom of the ring structure, and spring control units respectively provided at two inner sides of the ring structure, where
the PPG detection unit is configured to detect a PPG at a fingertip of a user;
the pressure sensing unit is configured to detect a contact pressure at the fingertip of the user;
the temperature sensing unit is configured to acquire a temperature at the fingertip of the user;
the MCU is connected to the PPG detection unit, the pressure sensing unit and the temperature sensing unit, and configured to calculate an APPG according to the PPG, the pressure and the temperature, and detect a BP according to the APPG; and
the spring control unit is connected to the pressure sensing unit, and configured to adjust the pressure sensing unit according to the pressure.

Further, the PPG detection unit includes a photoelectric sensor module; and the photoelectric sensor module is configured to detect the PPG at the fingertip of the user, and send the PPG to the MCU through an analog front-end (AFE) or an interface circuit;
the pressure sensing unit includes a pressure sensor; and the pressure sensor is configured to detect the contact pressure at the fingertip of the user, and send the pressure to the MCU through the AFE or the interface circuit; and
the temperature sensing unit includes a temperature sensor; and the temperature sensor is configured to acquire the temperature at the fingertip of the user, and send the temperature to the MCU through the AFE or the interface circuit.

Further, the BP detection apparatus further includes an inertial measurement unit (IMU); and the IMU is connected to the PPG detection unit, the pressure sensing unit, the temperature sensing unit and the MCU, and configured to detect whether data detected by each of the PPG detection unit, the pressure sensing unit and the temperature sensing unit contains interference data, and send interference-removed data to the MCU.

Further, the spring control unit includes a precision motor, a ratchet structure and a constant-force elastic module; the constant-force elastic module includes a constant force spring, a movable clamp and a fixed clamp; the precision motor drives the ratchet structure according to the pressure to rotate, thereby adjusting a length of the constant force spring; the movable clamp is sleeved on the constant force spring; the fixed clamp is sleeved on an upper portion of the movable clamp; the constant force spring includes a top connected to the ratchet structure, and a bottom connected to the bottom of the ring structure; and the fixed clamp and the movable clamp are fixed in the ring structure.

Further, the photoelectric sensor module includes a plurality of light-emitting diodes (LEDs) and a plurality of photodetectors (PDs); and the plurality of LEDs are centrosymmetric with respect to the plurality of PDs.

Further, the photoelectric sensor module includes a plurality of LEDs and one PD; and the plurality of LEDs are provided around the PD.

Further, calculating the APPG according to the PPG, the pressure and the temperature, and detecting the BP according to the APPG specifically include:
determining a cardiovascular state of the user with a cardiovascular state screening model according to the PPG;
selecting a corresponding BP prediction model from a BP prediction model set according to the cardiovascular state of the user;
calculating the APPG according to the PPG, the pressure and the temperature; and
detecting the BP with the selected BP prediction model according to the APPG.

Further, the cardiovascular state screening model is trained as follows:
constructing a training dataset, the training dataset including PPGs and BP data for different cardiovascular states, different genders and different ages, and the cardiovascular states including a normal, AF and coronary arteriosclerosis (CA);
dividing the PPGs into a first set of training data and a second set of training data;
extracting feature information from the first set of training data to obtain multiple types of feature information; and
training a machine learning model through the multiple types of feature information and the cardiovascular states corresponding to the PPGs to obtain the cardiovascular state screening model.

Further, the BP prediction model set is trained as follows:
training multiple deep learning models through the second set of training set in the different cardiovascular states and the corresponding BP data to obtain the BP prediction model set, the BP prediction model set including multiple BP detection models, and the BP detection models being configured to respectively detect BPs in the cardiovascular states.

Further, calculating the APPG according to the PPG, the pressure and the temperature specifically includes:
determining an adjustment amount of the spring control unit according to the pressure;
acquiring the PPG when the LEDs have a wavelength of λ₁ and a wavelength of λ₂ under the adjustment amount of the spring control unit
determining a scale factor according to the PPG;
determining a revised item of the scale factor according to the temperature; and
calculating the APPG according to the PPG, the scale factor and the revised item of the scale factor.

According to specific embodiments provided in the present disclosure, the present disclosure has the following technical effects:
(1) The BP detection apparatus based on an APPG provided by the present disclosure measures the vessel-rich finger with the ring, and achieves a simple structure and a simple operation. The obtained signal has a high SNR and a high accuracy. The present disclosure realizes continuous and dynamic real-time acquisition of the aortic BP of the human body, and has a high medical value and a broad market application prospect.
(2) With a multi-wavelength photoelectric detection sensor module, the present disclosure detects the PPG signal at different penetration depths. In combination with the motor, the constant force spring and the pressure sensor, the present disclosure realizes pressure control on the measured part. Meanwhile, in combination with the temperature of the detected part, the present disclosure removes an interference signal, and realizes direct measurement and tracking of the APPG signal.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 is a schematic view of a BP detection apparatus based on an APPG according to an embodiment of the present disclosure;

FIG. 2 is a schematic view of a ring structure in a BP detection apparatus based on an APPG according to an embodiment of the present disclosure;

FIG. 3 is a sectional view of a ring structure in a BP detection apparatus based on an APPG according to an embodiment of the present disclosure;

FIG. 4 is a schematic structural view of a constant-force elastic module in a BP detection apparatus based on an APPG according to an embodiment of the present disclosure;

FIG. 5 is a schematic view of feedback control of a constant-force elastic module in a BP detection apparatus based on an APPG according to an embodiment of the present disclosure;

FIG. 6 is a schematic view of a bottom of a ring structure in a BP detection apparatus based on an APPG according to an embodiment of the present disclosure;

FIG. 7 illustrates an implementation principle of APPG detection of a BP detection apparatus based on an APPG according to an embodiment of the present disclosure;

FIG. 8 illustrates a waveform of an APPG, a pressure and a temperature of a BP detection apparatus based on an APPG according to an embodiment of the present disclosure;

FIG. 9 illustrates an implementation principle of APPG, pressure and temperature detection of a BP detection apparatus based on an APPG according to an embodiment of the present disclosure;

FIG. 10 illustrates a principle of APPG signal detection of a BP detection apparatus based on an APPG according to an embodiment of the present disclosure;

FIG. 11 illustrates a learning and training flowchart of a population model of a BP detection apparatus based on an APPG according to an embodiment of the present disclosure;

FIG. 12A-12B are schematic views of feature extraction of a BP detection apparatus based on an APPG according to an embodiment of the present disclosure;

FIG. 13 is a cardiovascular state screening flowchart of a BP detection apparatus based on an APPG according to an embodiment of the present disclosure;

FIG. 14 is a schematic view of construction for a population model of a BP detection apparatus based on an APPG according to an embodiment of the present disclosure; and

FIG. 15 is a schematic view of construction for a transfer learning model of a BP detection apparatus based on an APPG according to an embodiment of the present disclosure.

In the figures:
1: photoelectric sensor module;
1-1: λ₁-wavelength LED light source;
1-2: λ₂-wavelength LED light source;
1-3: PD;
1-1-1: PPG received by the λ₁-wavelength LED light source at 1-3;
1-2-1: PPG received by the λ₂-wavelength LED light source at 1-3;
1-12-1: APPG received by the λ₁-wavelength LED light source and the λ₂-wavelength LED light source;
2: Temperature sensor;
3: Pressure sensor;
4: Constant-force elastic module;
4-1: Constant force spring;
4-2: Movable clamp;
4-3: Fixed clamp;
4-4: Top threaded hole;
4-5: Bottom threaded hole;
4-6: Top connecting wire;
4-7: Bottom connecting wire;
5: Battery;
6-1: Ring bottom right threaded hole;
6-2: Ring bottom left threaded hole;
6-3: MCU;
6-4: Electronic element; and
6-5: Sensor spring structure.

### DETAILED DESCRIPTION OF THE EMBODIMENTS

The technical solutions in the embodiments of the present disclosure are clearly and completely described below with reference to the drawings in the embodiments of the present disclosure. Apparently, the described embodiments are merely a part rather than all of the embodiments of the present disclosure. All other embodiments obtained by those skilled in the art based on the embodiments of the present disclosure without creative efforts shall fall within the protection scope of the present disclosure.

An objective of the present disclosure is to provide a BP detection apparatus based on an APPG, to improve an accuracy of BP prediction.

In order to make the above objective, features and advantages of the present disclosure clearer and more comprehensible, the present disclosure will be further described in detail below in combination with accompanying drawings and particular implementations.

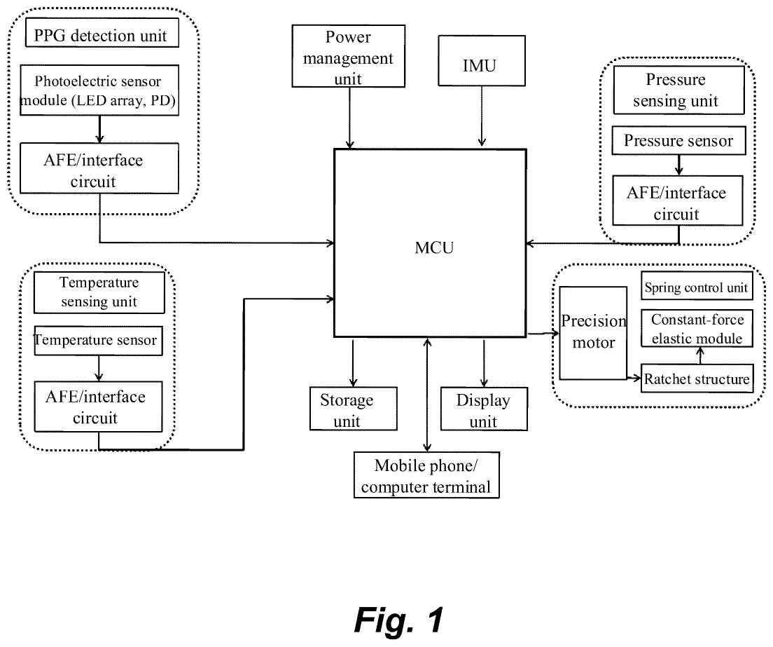

As shown in FIG. 1 to FIG. 6, a BP detection apparatus based on an APPG includes a ring structure, a PPG detection unit, a temperature sensing unit, a pressure sensing unit and an MCU provided at a bottom of the ring structure, and spring control units respectively provided at two inner sides of the ring structure.

The PPG detection unit is configured to detect a PPG at a fingertip of a user.

The pressure sensing unit is configured to detect a contact pressure at the fingertip of the user.

The temperature sensing unit is configured to acquire a temperature at the fingertip of the user.

The MCU is connected to the PPG detection unit, the pressure sensing unit and the temperature sensing unit. The MCU is configured to calculate an APPG according to the PPG, the pressure and the temperature, and detect a BP according to the APPG. The MCU includes a Bluetooth low energy (BLE) protocol stack. The MCU is configured to control each detection unit to acquire signal data, perform analysis, feature extraction and BP calculation on a signal, and send a BP detection result to a terminal through the BLE protocol stack.

The spring control unit is connected to the pressure sensing unit. The spring control unit is configured to adjust the pressure sensing unit according to the pressure.

The BP detection apparatus further includes an IMU. The IMU is connected to the PPG detection unit, the pressure sensing unit, the temperature sensing unit and the MCU. The IMU is configured to detect whether data detected by each of the PPG detection unit, the pressure sensing unit and the temperature sensing unit contains interference data, and send interference-removed data to the MCU.

The BP detection apparatus further includes a power management unit and a storage unit. The power management unit is configured to supply power to a power consumption unit. The storage unit is configured to store local data.

The PPG detection unit includes a photoelectric sensor module 1. The photoelectric sensor module 1 is configured to detect the PPG at the fingertip of the user, and send the PPG to the MCU 6-3 through an AFE or an interface circuit.

The pressure sensing unit includes a pressure sensor 3. The pressure sensor 3 is configured to detect the contact pressure at the fingertip of the user, and send the pressure to the MCU 6-3 through the AFE or the interface circuit.

The temperature sensing unit includes a temperature sensor 2. The temperature sensor 2 is configured to acquire the temperature at the fingertip of the user, and send the temperature to the MCU 6-3 through the AFE or the interface circuit.

The spring control unit includes a precision motor, a ratchet structure and a constant-force elastic module 4. The precision motor drives the ratchet structure according to the pressure to rotate, thereby adjusting a length of a constant force spring 4-1. The constant-force elastic module 4 includes the constant force spring 4-1, a movable clamp 4-2 and a fixed clamp 4-3. The movable clamp 4-2 is sleeved on the constant force spring 4-1. The fixed clamp 4-3 is sleeved on an upper portion of the movable clamp 4-2. The constant force spring 4-1 includes a top connected to the ratchet structure, and a bottom connected to the bottom of the ring structure. The fixed clamp 4-3 and the movable clamp 4-2 are fixed in the ring structure.

Each unit in the BP detection apparatus is a detachable structure, and is assembled modularly. With the detachable manner, portions such as a sensing portion, a pressure control portion and a power control portion can be separated.

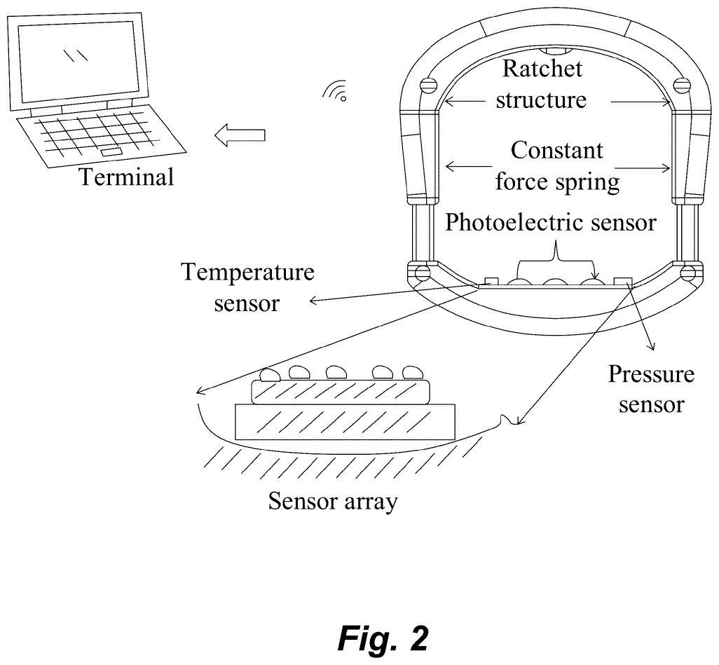

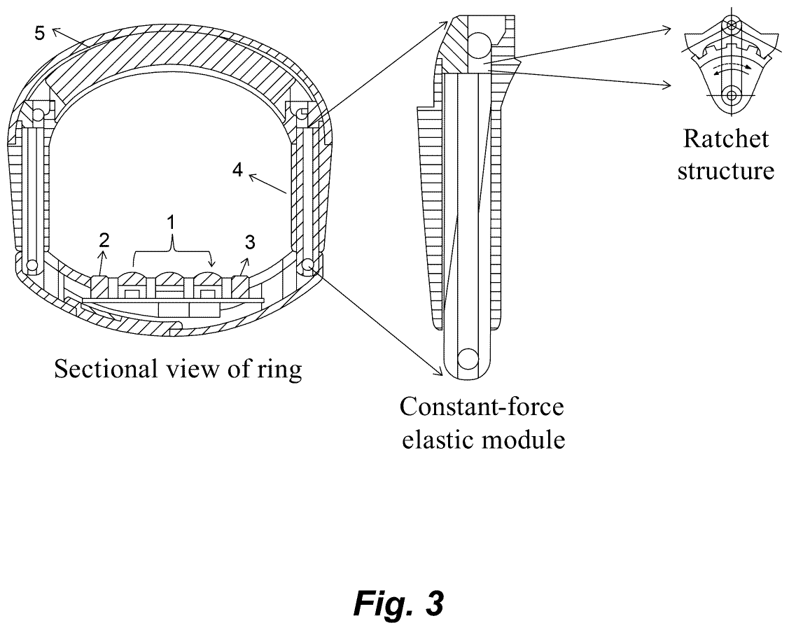

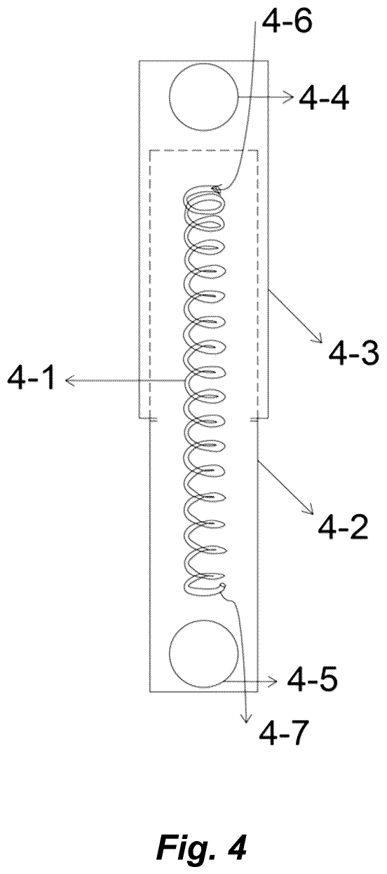

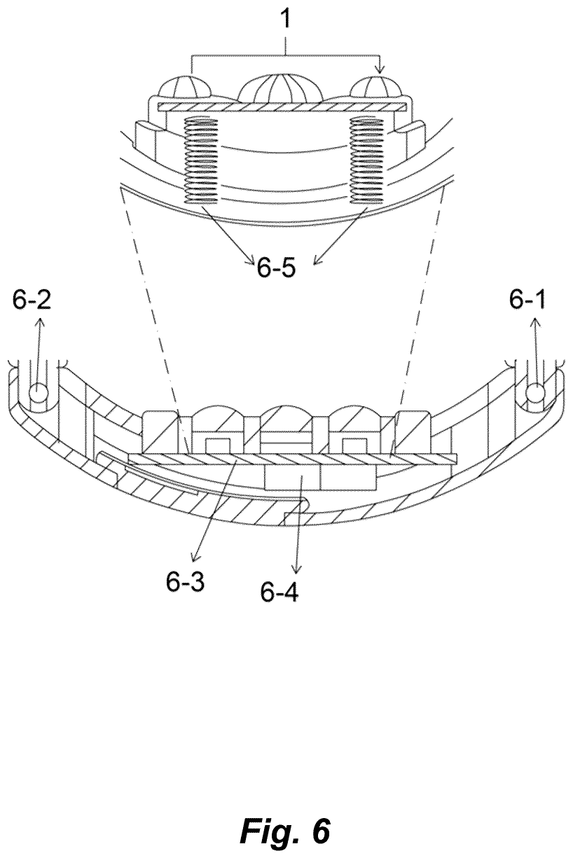

As shown in FIG. 2, FIG. 3, FIG. 4 and FIG. 6, the pressure sensor 3, the photoelectric sensor module 1 and the temperature sensor 2 form a sensor detection module. The sensor detection module is located on a finger pulp, and is connected to the constant-force elastic modules 4 through a ring bottom right threaded hole 6-1, a ring bottom left threaded hole 6-2 as well as bottom threaded holes 4-5 of the constant-force elastic modules.

The photoelectric sensor module 1 may be composed of a plurality of LEDs and a plurality of PDs. The plurality of LEDs (such as 1-1 and 1-2) may surround one PD (such as 1-3) to form a circular array. The LEDs (such as 1-1 and 1-2) may also be centrosymmetric with respect to the PD (such as 1-3).

The ratchet structure and a battery 5 are mainly provided on a top of the ring structure. The ring structure is connected to the constant-force elastic modules 4 through top left and right threaded holes of the ring structure as well as top threaded holes 4-4 of the constant-force elastic modules.

The constant-force elastic modules 4 are respectively located at two sides of the ring, and arranged symmetrically. The constant-force elastic modules at a left side and a right side of the ring are structurally the same. The constant-force elastic module 4 is connected to the ratchet structure through a top connecting wire 4-6. The constant-force elastic module 4 is connected to the sensor detection module through a bottom connecting wire 4-7. When the precision motor drives the ratchet structure to rotate, the ratchet structure controls the length of the constant force spring 4-1, thereby adjusting a pressure between the sensor detection module and the finger pulp. In addition, for a sensor spring structure 6-5 under the sensor detection module, in order to keep full contact between the sensor detection module and the finger pulp, a length of the sensor spring structure 6-5 is adjusted secondarily as the length of the constant force spring 4-1 changes.

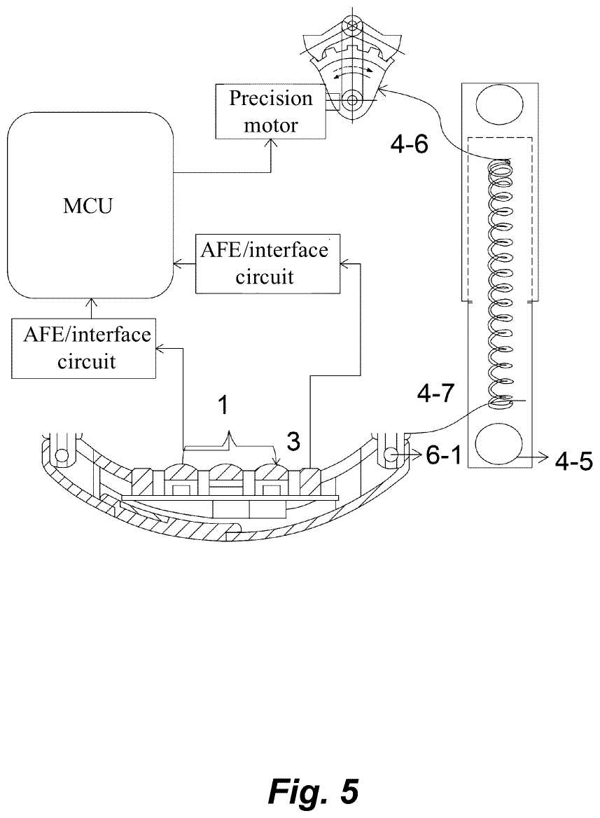

FIG. 5 illustrates an implementation of a pressure adjusting function. The MCU 6-3 detects a measured pressure of the pressure sensor 3 through the AFE or the interface circuit of the pressure sensing unit. The MCU 6-3 detects a measured PPG signal of the photoelectric sensor module 1. When the ring is just worn, a pressure is labeled as F₀, and a corresponding PPG signal is labeled as PPG₀. The MCU 6-3 controls the precision motor to rotate, thereby driving the ratchet structure to rotate clockwise for N steps (N≥1). The ratchet structure pulls the constant force spring 4-1 through the top connecting wire 4-6, thereby pulling the whole sensor detection module. By this time, an elastic potential energy of the sensor spring structure 6-5 increases. A pressure F₁ measured by the pressure sensor 3 and a corresponding PPG₁ are labeled. An alternating-current (AC) amplitude AC_PPG₀ corresponding to the PPG₀ is compared with an AC amplitude AC_PPG₁ corresponding to the PPG₁. If AC_PPG₀>AC_PPG₁, the ratchet structure rotates anticlockwise for M steps (N≥M≥0). On the contrary, if AC_PPG₀≤AC_PPG₁, the ratchet structure continues to rotate clockwise for N steps. By cyclically operating this method, an appropriate position i of the ratchet structure is determined, a value Fᵢ of the pressure sensor is labeled, and an AC_PPGᵢ at the present position is maximized.

Therefore, according to the pressure measured by the pressure sensor, the spring control unit drives the ratchet structure through the precision motor to rotate, thereby adjusting the length of the constant force spring 4-1 and adjusting the sensor detection module. That is, the pressure between the pressure sensor 3 and the finger pulp is adjusted by pulling or compressing the pressure sensor 3.

In the BP detection apparatus, an electronic element 6-4 such as a storage unit, a resistor and a capacitor is integrated to the bottom of the ring.

The BP detection apparatus based on an APPG provided by the present disclosure measures the vessel-rich finger with the ring, and achieves a simple structure and a simple operation. The obtained signal has a high SNR and a high accuracy. The present disclosure realizes continuous and dynamic real-time acquisition of the aortic BP of the human body, and has a high medical value and a broad market application prospect.

The MCU in the BP detection apparatus based on an APPG provided by the present disclosure is specifically implemented as follows:
Step 1: A cardiovascular state of the user is determined with a cardiovascular state screening model according to the PPG.
Step 2: A corresponding BP prediction model is selected from a BP prediction model set according to the cardiovascular state of the user.
Step 3: The APPG is calculated according to the PPG, the pressure and the temperature.
Step 4: The BP is detected with the selected BP prediction model according to the APPG. The cardiovascular state screening model in the step 1 is trained as follows:
Step 11: A training dataset is constructed, the training dataset including PPGs and BP data for different cardiovascular states, different genders and different ages, and the cardiovascular states including a normal, AF and CA.
Step 12: The PPGs is divided into a first set of training data and a second set of training data.
Step 13: Feature information is extracted from the first set of training data to obtain multiple types of feature information.
Step 14: A machine learning model is trained through the multiple types of feature information and the cardiovascular states corresponding to the PPGs to obtain the cardiovascular state screening model.

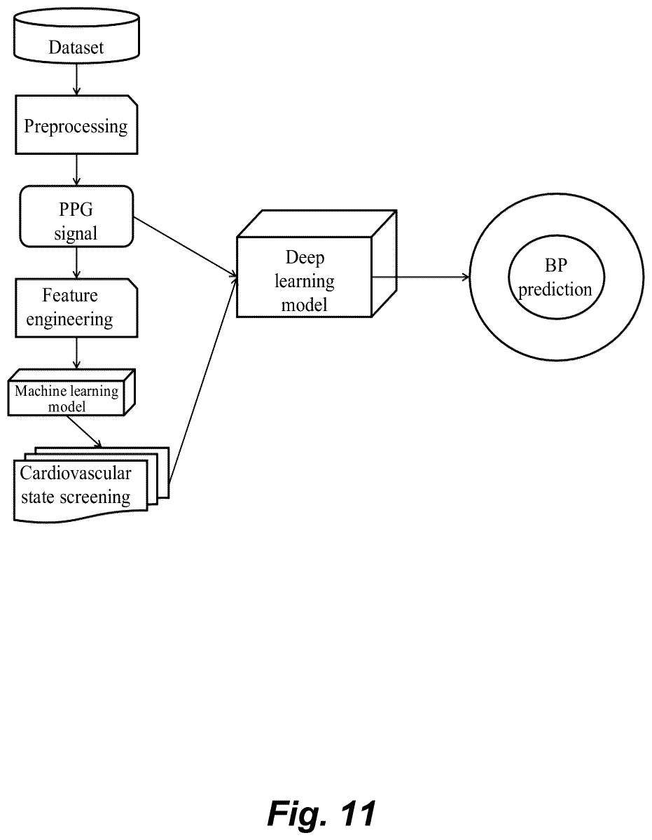

In a specific embodiment, as shown in FIG. 11, a disclosed dataset including arterial blood pressures (ABPs), PPGs, cardiovascular states, ages and genders at the same time is selected, such as a Multiparameter Intelligent Monitoring in Intensive Care (MIMIC) dataset or a dataset disclosed by the Guilin University, for data preprocessing. A minimum length required by each of the PPG and the ABP is set as 5 min, and all records with a short length are deleted. A baseline drift and a high-frequency noise in the PPG signal are removed with a fourth-order Butterworth band-pass filter. The PPG signal has a passband frequency of 0.5-8 Hz. A spike noise in the ABP signal is eliminated with a Hampel filter.

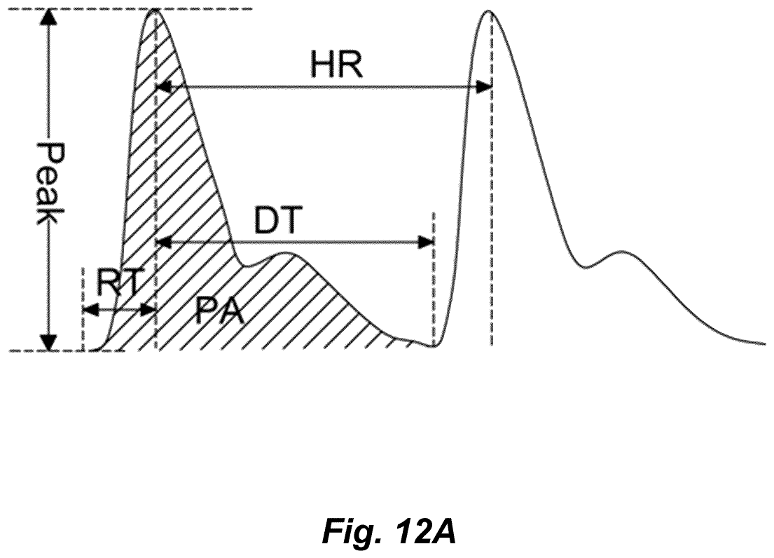

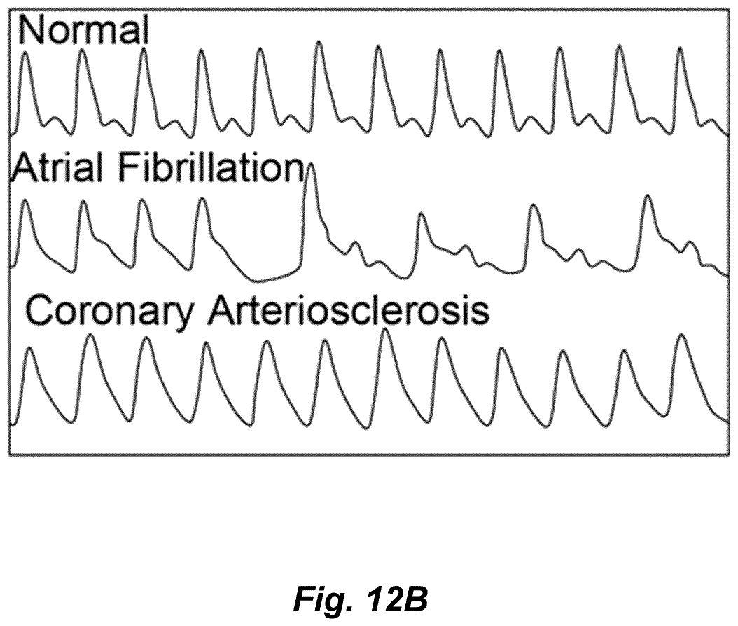

The PPGs divided based on different cardiovascular states are constructed through the disclosed dataset. The disclosed dataset includes data subsets {NORMAL}, {AF} and {CA} for the cardiovascular states, the genders, the ages and the BPs. The preprocessed PPG signal is divided into two types (namely the first set of training data and the second set of training data). The first type is used for feature engineering to extract features. The features serve as an input of the machine learning model to train model parameters and output a screening classification result on the cardiovascular state. The second type serves as an input of the deep learning model for BP prediction. Extracted feature information in the feature engineering includes, but is not limited to, a heart rate (HR), a peak value Peak, rise time RT, down time DT, a pulse area PA, a heart rate variability (HRV), etc. As shown in FIG. 12A, the extracted feature information further includes a frequency domain, a time-frequency domain, a nonlinear feature such as a sample entropy, etc. The feature information is used for training, verification and testing in classification of the cardiovascular state. After a tested accuracy rate is greater than 90%, model parameters are fixed, thereby screening the cardiovascular state. Meanwhile, existing evidences show that features of the PPG, including an intensity, a pattern, a rhythm and a rate, change with a geometric shape and a mechanical property of the vessel. PPG patterns corresponding to different cardiovascular states are significantly different. As shown in FIG. 12B, the PPG pattern of a person with the normal cardiovascular state includes an obvious regular dicrotic notch (DN). The PPG pattern of a person with the abnormal cardiovascular state is significantly different in the PA, DN, HR and HRV. In view of this, different classification models can be designed to classify and assess the cardiovascular state.

The classification models include:
1) Bayes classifier: Since a Bayes' theorem serves as a core in the algorithm, Bayesian classification is called. According to a Bayesian decision theory, through known dependent probabilities, a misjudgment loss is used to select an optimal category for classification.
2) Decision tree classifier: In the decision tree algorithm, samples in a training set of multidimensional features are allocated to different categories. In other words, the samples in the training set are projected. Corresponding category labels are assigned to projected samples in the training set. In the decision tree algorithm, a recursive process is used to indicate classification and regression. In each time, an optimal feature in a feature set is selected. The training set is classified and regressed according to the selected optimal feature. This ensures that classification and regression for each sample in the training set are optimal. In case of numerous features for the samples in the training set, the features are selected when the decision tree is generated. The strong features are remained to classify and regress the trained samples in the training set, while the weak features are removed. The whole recursive process is shaped as a tree, so the decision tree algorithm is called. The decision tree algorithm aims to construct the highly accurate and less complicated decision tree. The construction of the decision tree includes generation and pruning of the decision tree. The decision tree is generated with the samples in the training set. A top node of the decision tree serves as a root node. Each branch is viewed as a decision node, and represents an attribute of a to-be-classified object. Each node represents a possible classification process. When the decision tree is traversed, different selections of each decision node lead to different output branches. At last, the result is output from a leaf node. The traversing process is to solve the classification problem with the decision tree. A plurality of attributes are used to classify the samples in the training set. The pruning of the decision tree is to check and correct the generated decision tree. By simplifying the decision tree, the decision tree has a better generalization capability and can be adapted for more solutions.
3) Random forest (RF) classifier: RF is an optimized version of Bagging based on a tree model. Through a plurality of trees, the RF solves the weak generalization capability of the decision tree. For a same batch of data, only one tree can be generated with a same algorithm. With the Bagging policy, different datasets can be generated. The Bagging policy comes from bootstrap aggregation. Resampling is performed on a sample set (assuming that the sample set includes N data points) to select Nb samples (sampling with replacement, there are still N data points for the samples). On all samples, a classifier (ID3\C4.5\CART\SVM\LOGISTIC) is established for the N samples. The above two steps are repeated m times to obtain m classifiers. According to poll results of the m classifiers, the category of the data is determined.

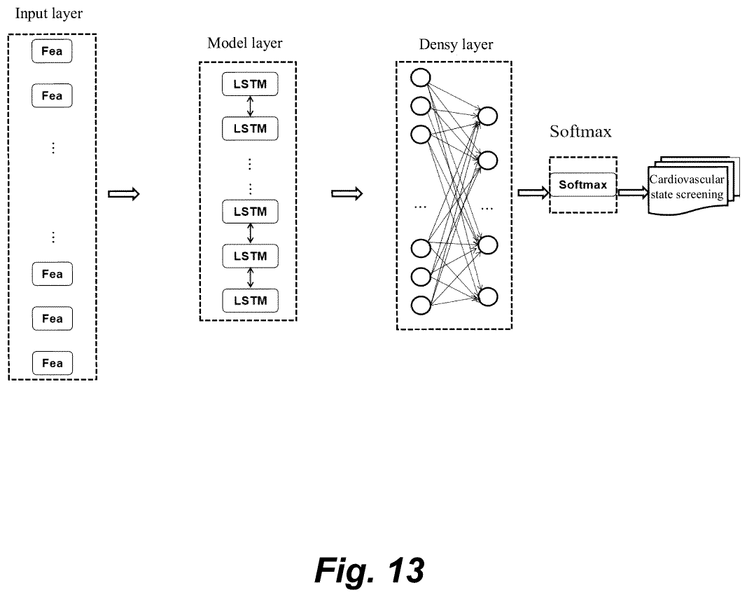

In addition, FIG. 13 illustrates a typical classification example. Multidimensional features generated by the feature engineering, including a time domain, a frequency domain, a time-frequency domain and a nonlinear feature such as a sample entropy, are taken as an input layer. In order to prevent overfitting, when age data of the PD is taken as feature data in cardiovascular state classification for training, a data synthesis policy is used. A random Gaussian noise is added to the age feature, and data with a same age is expanded to a ±3 range or a ±5 range. A multi-layer feedback network is constructed with a long short term memory (LSTM) or a bidirectional LSTM (Bi-LSTM) on a model layer. A Densy layer and a softmax layer are provided to output a classification result. The model layer may also be an RF model, a support vector machine (SVM) model, etc. The table below preliminarily shows an average accuracy of the models in response to Normal, AF and CA cardiovascular states.

TABLE 1
Classification accuracy

| | Precision | Recall | Accuracy | F1 |
|---|---|---|---|---|
| SVM | 0.80 | 0.80 | 0.80 | 0.80 |
| RF | 0.93 | 0.93 | 0.93 | 0.93 |
| Bi-LSTM | 0.95 | 0.95 | 0.95 | 0.95 |

The present disclosure uses the RF classifier as the machine learning model to screen the cardiovascular state. In actual applications, the appropriate classifier can be selected according to computing resources.

The BP prediction model set in the step 2 is trained as follows:

Multiple deep learning models are trained through the second set of training set in the different cardiovascular states and the corresponding BP data to obtain the BP prediction model set, the BP prediction model set including multiple BP detection models, and the BP detection models being configured to respectively detect BPs in the cardiovascular states.

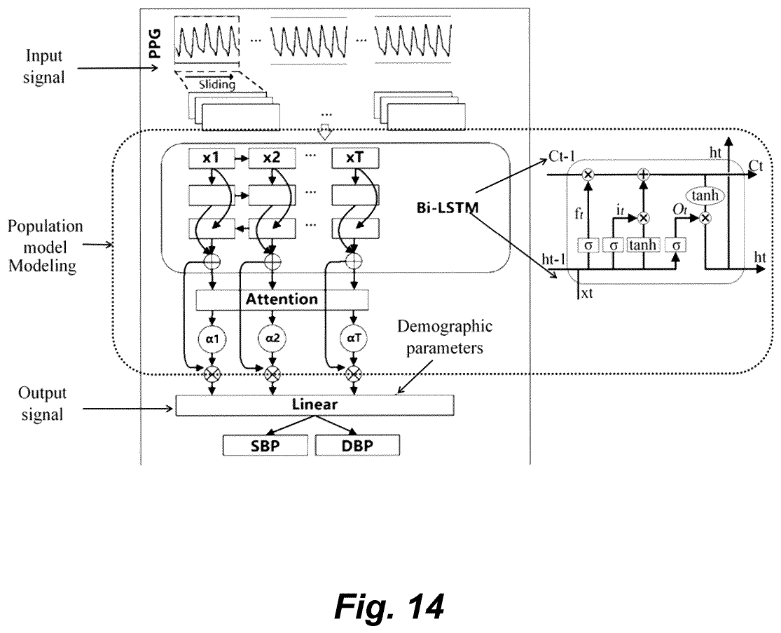

In a specific embodiment, as shown in FIG. 14, for the classification result of each cardiovascular state screening model, the BP prediction model set is established based on a deep learning method. The present disclosure provides a novel model architecture, namely a recurrent-attention neural network (LSTM-AT) based on an LSTM, an attention mechanism and a fully connected (FC) neural network. A PPG signal segment within a certain time period serves as an input signal of the model. By taking a PPG segment of data in the data subset as the input signal, and a BP as a label signal to train the deep learning BP prediction model, the BP prediction model set for different data subsets is constructed. Parameters of the BP prediction model are fixed and taken as population model coefficients. The input of the BP prediction model is the PPG data segment. Through the Bi-LSTM layer and the Attension layer, an important feature vector is weighted. The output vector is combined with the age, the gender and other demographic information. The BP is predicted through the FC layer. The BP prediction model employs a Bahdanau attention:

$${c}_{i} = {\sum }_{i = 0}^{T} {a}_{i} {h}_{ti} ,$$

where, cᵢ keeps all hidden information output from a previous layer and represents a weighted sum between an attention weight vector aᵢ and an attention weight vector hₜᵢ, aᵢ is normalized with a softmax function, and hₜᵢ represents hₜ at time i.

As a special recurrent neural network (RNN), the LSTM can effectively solve gradient vanishing and bursting problems in the training. FIG. 14 illustrates a structural view of an LSTM unit. Compared with the RNN, the LSTM has two transmission states: Ct (a cellular state) and ht (a hidden state). The Ct changes very slowly. Different segments correspond to different ht, namely the LSTM and the short-term memory (STM). Internally, the LSTM transmits information from one time step to another time step through a forget gate, an input gate and an output gate.

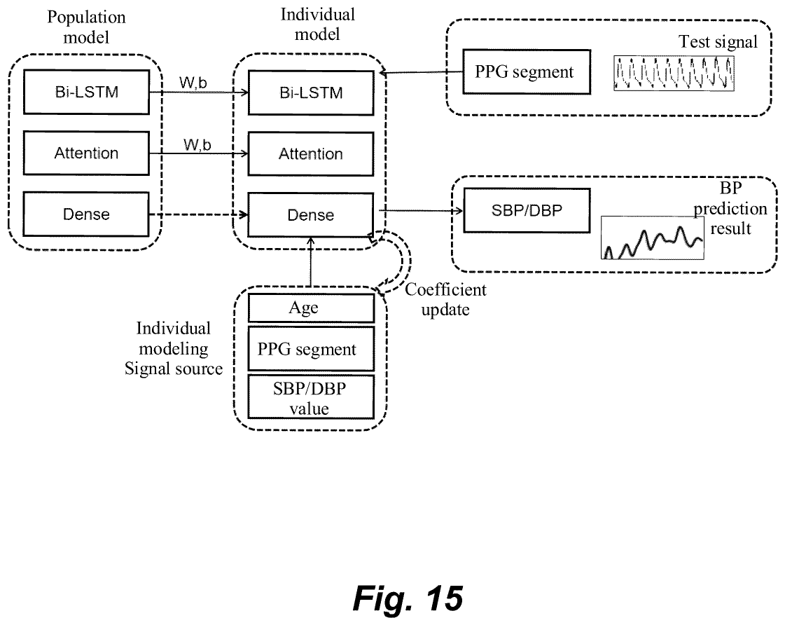

Transfer learning aims to store knowledge acquired from a source domain to a target domain. The problem usually involves a small number of data samples to train the model. Based on the novel model architecture, namely the recurrent-attention neural network (LSTM-AT) based on the LSTM, the attention mechanism and the FC neural network, the present disclosure acquires PPG data and BP data from a large disclosed dataset to preliminarily train parameters. In use of the individual, new data is used to adjust parameters on a last layer of the individual model. This can greatly reduce the number of training sets required by the new data, as shown in FIG. 15.

The step 3 specifically includes:
Step 31: An adjustment amount of the spring control unit is determined according to the pressure.
Step 32: The PPG when the LEDs have a wavelength of λ₁ and a wavelength of λ₂ under the adjustment amount of the spring control unit is acquired.
Step 33: A scale factor is determined according to the PPG.
Step 34: A revised item of the scale factor is determined according to the temperature.
Step 35: The APPG is calculated according to the PPG, the scale factor and the revised item of the scale factor.

The PPG detected by the PPG detection unit includes the PPG when the LEDs have the wavelength of λ₁ under the adjustment amount of the spring control unit, and the PPG when the LEDs have a wavelength of λ₂ under the adjustment amount of the spring control unit.

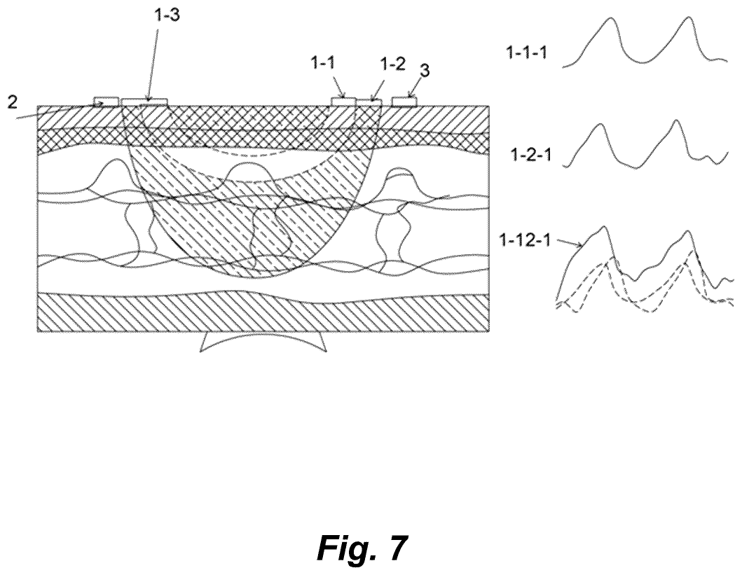

In a specific embodiment, the PD 1-3, the LED 1-1 and the LED 1-2 are used as an example. The LED 1-1 has the wavelength of 2, and the LED 1-2 has the wavelength of λ₁, λ₁≥λ₂. As can be seen, the light source with the wavelength of λ₁ has a large penetration depth, and photons in a propagation path involve a volume change signal of the capillary and a volume change signal of the arteriole. The light source with the wavelength of λ₂ has a small penetration depth, and photons in a propagation path mainly involve a volume change of the capillary. In this case, since the photons are mainly propagated in skin tissues in diffuse reflection, and are easily affected by tissue fluids and vein fluctuations, it is hard to accurately acquire a signal on a specific layer. As shown in FIG. 7, the PPG signal detected at the PD is a compound signal. 1-1-1 is the PPG received by the λ₁-wavelength LED light source at the PD 1-3. 1-2-1 is the PPG received by the λ₂-wavelength LED light source at the PD 1-3. 1-12-1 is the APPG synthesized by the λ₁-wavelength LED light source and the λ₂-wavelength LED light source.

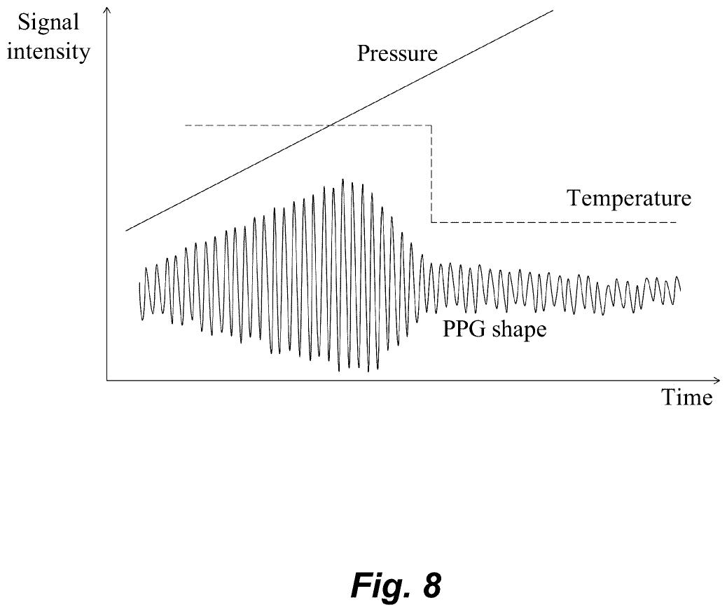

In addition to the propagation path of the photons, the PPG signal is further affected by a pressure signal and a temperature signal. As shown in FIG. 8, as the pressure increases, the AC amplitude of the PPG increases first and then decreases, and the arteriole experiences a process from load removal to obstruction. As the temperature decreases, the AC amplitude of the PPG decreases. Principally, when the temperature decreases, the vessel causes contraction, with a smaller inner diameter, a larger peripheral resistance, and less perfusion.

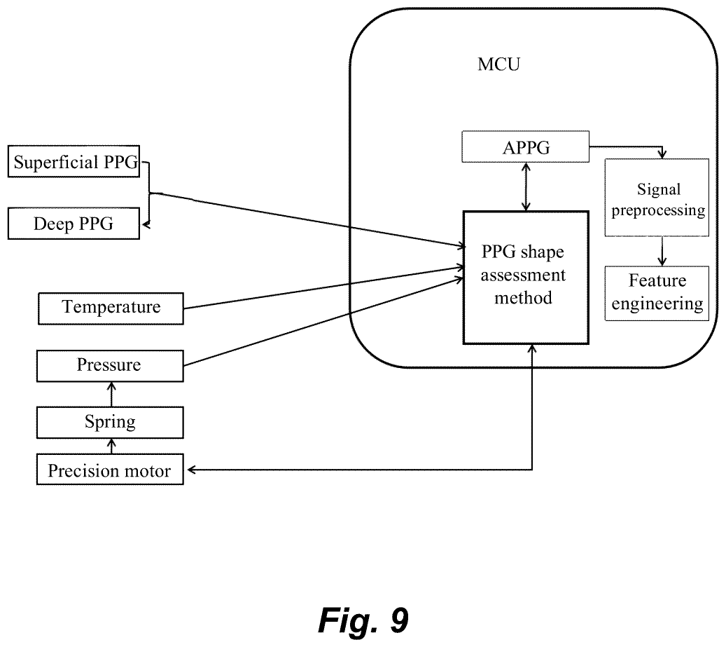

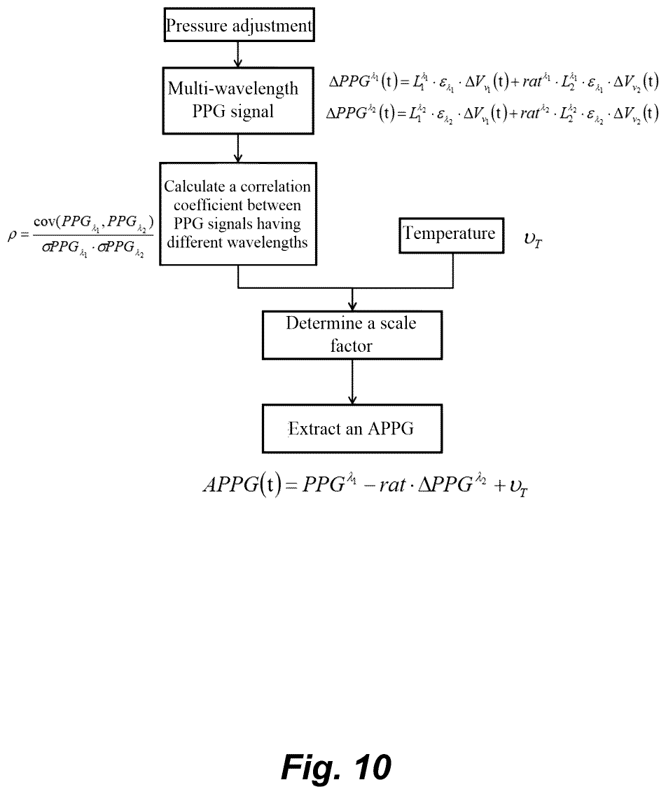

As shown in FIG. 9 and FIG. 10, the value Fᵢ of the pressure sensor, the temperature Tᵢ, the superficial PPG and the deep PPG serve as the input signal, and the APPG signal serves as the output signal.

The LED 1-2 has the wavelength of λ₁. The penetration path of the photons having the wavelength of λ₁ simply includes a capillary portion L₁^{λ}^{₁}. A volume change of the capillary portion over time t can be expressed as ΔV_{ν}_{₁}(t) According to the Beer-Lambert law, for the liquid with the penetration path being L₁^{λ}^{₁}, the volume change being ΔV_{ν}_{₁}(t) and a difference between an absorption coefficient and a background absorption coefficient being ε_{λ}_{₁}, a change of a light intensity at the PD can be expressed as L₁^{λ}^{₁}·ε_{λ}_{₁}·ΔV_{ν}_{₁}(t). Likewise, due to the large penetration depth, a change of a light intensity of an arteriole-penetrating portion at the PD can be expressed as L₂^{λ}^{₁}·ε_{λ}_{₁}·ΔV_{ν}_{₂}(t). Considering that a ratio of the photons of the arteriole-penetrating portion is affected by the skin structure and the body temperature, rat^{λ}^{₁} is introduced to correct the coefficient. The PPG received by the λ₁-wavelength light source at the PD can be specifically expressed by:

$${PPG}^{{λ}_{1}} ( t ) = {L}_{1}^{{λ}_{1}} \cdot {ε}_{{λ}_{1}} \cdot Δ {V}_{{v}_{1}} ( t ) + {rat}^{{λ}_{1}} \cdot {L}_{2}^{{λ}_{1}} \cdot {ε}_{{λ}_{1}} \cdot Δ {V}_{{v}_{2}} ( t ) \qquad ( 1 )$$

By the same reasoning, the LED 1-1 has the wavelength of λ₂. The penetration path of the photons having the wavelength of λ₂ simply includes a capillary portion L₁^{λ}^{₂}. A volume change of the capillary portion over time t can be expressed as ΔV_{ν}_{₁}(t) According to the Beer-Lambert law, for the liquid with the penetration path being L₁^{λ}^{₂}, the volume change being ΔV_{ν}_{₁}(t) and a difference between an absorption coefficient and a background absorption coefficient being ε_{λ}_{₂}, a change of a light intensity received at the PD can be expressed as L₁^{λ}^{₂}·ε_{λ}_{₂}·ΔV_{ν}_{₁}(t). Likewise, due to the small penetration depth, only a small part of the photons penetrate through the arteriole, and a change of a light intensity received at the PD can be expressed as L₂^{λ}^{₂}·ε_{λ}_{₂}·ΔV_{ν}_{₂}(t). Considering that a ratio of the photons of the arteriole-penetrating portion is affected by the skin structure and the body temperature, rat^{λ}^{₂} is introduced to correct the coefficient. The PPG received by the λ₂-wavelength light source at the PD can be specifically expressed by:

$${PPG}^{{λ}_{2}} ( t ) = {L}_{1}^{{λ}_{2}} \cdot {ε}_{{λ}_{2}} \cdot Δ {V}_{{v}_{1}} ( t ) + {rat}^{{λ}_{2}} \cdot {L}_{2}^{{λ}_{2}} \cdot {ε}_{{λ}_{2}} \cdot Δ {V}_{{v}_{2}} ( t ) \qquad ( 2 )$$

When the coefficient is calculated, λ₁>>λ₂. The λ₁-wavelength light source has the large penetration depth, and photons in the propagation path involve the volume change signal of the capillary and the volume change signal of the arteriole. The λ₂-wavelength light source has the small penetration depth, and photons in the propagation path mainly involve the volume change of the capillary. In this case, L₁^{λ}^{₁}·ε_{λ}_{₁}·ΔV_{ν}_{₁}(t), L₂^{λ}^{₁}·ε_{λ}_{₁}·ΔV_{ν}_{₂}(t), L₁^{λ}^{₂}·ε_{λ}_{₂}·ΔV_{ν}_{₁}(t) and L₂^{λ}^{₂}·ε_{λ}_{₂}·ΔV_{ν}_{₂}(t) each are a constant, and rat^{λ}^{₁}>>rat^{λ}^{₂}. In order to simplify the model calculation, the constant term is ignored, rat^{λ}^{₂}>=0 and rat^{λ}^{₁}=1, thereby obtaining the following Eq.:

$$APPG ( t ) = [ {PPG}^{{λ}_{1}} - rat ( t ) {PPG}^{{λ}_{2}} ] ∝ Δ {V}_{{v}_{2}} ( t ) \qquad ( 3 )$$

As can be seen, by seeking a differential between the light sources having different wavelengths, the APPG can be obtained. Hence, there is a need to clarify a calculation method of the scale factor rat (t). The calculation of the scale factor rat (t) is associated with penetration depths of different light sources at the time t, and contraction and expansion of arteries, veins and tissues. Specifically:
1) The rat has a value of 0-10, and increases at 0.01. For each value of the rat, a set of signal matrix PPG=PPG_{λ}_{₂}−rat*PPG_{λ}_{₁} is calculated.
2) A cross-correlation coefficient between the PPG and the PPG_{λ}_{₁} is calculated by:

$$ρ = \frac{cov ( {PPG}_{{λ}_{1}} , PPG )}{σ {PPG}_{{λ}_{1}} \cdot σ PPG} . \\ cov ( {PPG}_{{λ}_{1}} , PPG ) = \frac{{\sum }_{i = 1}^{n} ( {PPG}_{{λ}_{1}} - \overline{{PPG}_{{λ}_{1}}} ) * ( PPG - \overline{PPG} )}{n - 1} ,$$

σ is a variance, n is a number of points, and P̅P̅G̅_̅{̅λ̅}̅_̅{̅₁̅}̅, P̅P̅G̅ is an average for the n points. When the cross-correlation coefficient is >0.5 and the stable time t (t>3 s) is maintained, the rat corresponding to the present data is the value of the scale factor rat.
3) An impact of the temperature on the PPG amplitude is assessed through multiple experiments. By keeping factors such as the HR constant, a change trend of the PPG amplitude over the temperature is recorded. ν_{T} represents the revised item of the temperature for the PPG amplitude.
4) Though the rat determined in the step 2), and the ν_{T} determined in the step 3), the value of the APPG is determined by:

$$APPG ( t ) = {PPG}^{{λ}_{1}} - rat \cdot {PPG}^{{λ}_{2}} + {υ}_{T} \qquad ( 4 )$$

PPG^{λ}^{₁} and PPG^{λ}^{₂} are respectively an amplitude measured when the wavelength is λ₁ and an amplitude measured when the wavelength is λ₂, and ν_{T} represents the revised item of the temperature for the PPG amplitude, and can be taken as a compensation to obtain a more accurate APPG waveform. Through the above steps, the PPG signal at the arteriole can be obtained for subsequent cardiovascular state assessment and BP prediction.

The present disclosure keeps the population model (namely the BP prediction model set) and the coefficient unchanged in the MCU. The MCU detects the multi-channel PPGs through the PPG detection unit, controls the ratchet structure through the motor, changes the length of the constant force spring and maintains the constant acting force Fᵢ. At the Fᵢ, the AC amplitude of the PPG is maximized, the temperature is labeled, and the cross-correlation coefficient between the multi-channel PPGs is calculated. When the cross-correlation coefficient is >0.5 and the stable time t (t>3 s) is maintained, the scale factor rat is determined, and the APPG is calculated according to APPG(t)=PPG^{λ}^{₁}−rat·PPG^{λ}^{₂}+ν_{T}. By calibrating data of the individual once, and updating the coefficient on the last layer of the BP prediction model through transfer learning, the personalized BP detection is realized. That is, the cardiovascular states are screened through the feature engineering and divided into different states s such as NORMAL, AF and CA. From the BP prediction model set, BP prediction model coefficients wᵢ corresponding to the different states sᵢ are selected for the BP prediction. sᵢ represents an ith cardiovascular state, and wᵢ represents a parameter of the BP prediction model corresponding to the ith cardiovascular state. During modeling of the individual model (namely the BP prediction model), the coefficient on the last layer of the BP prediction model is updated through the PPG signal, the age, the gender, the previous history and other input parameters of the individual model within N seconds. The transfer learning model on the individual indicates that the BP predicted with the model in the present disclosure meets class-A requirements of the British Hypertension Society (BHS) standard, as shown in Table 2 below.

TABLE 2
Comparison between a test result of the transfer
learning model and the BHS standard

| Cumulative Error | | ≤5 mmHg | ≤10 mmHg | ≤15 mmHg | Grade |
|---|---|---|---|---|---|
| BHS | | 60% | 85% | 95% | A |
| | | 50% | 75% | 90% | B |
| | | 40% | 65% | 85% | C |
| Normal | SBP | 84% | 96% | 99% | A |
| | DBP | 92% | 98% | 99% | A |
| AF | SBP | 82% | 93% | 97% | A |
| | DBP | 96% | 100% | 100% | A |
| CA | SBP | 68% | 88% | 95% | A |
| | DBP | 85% | 97% | 99% | A |

With a multi-wavelength photoelectric detection sensor module, the present disclosure detects the PPG signal at different penetration depths. In combination with the motor, the constant force spring and the pressure sensor, the present disclosure realizes pressure control on the measured part. Meanwhile, in combination with the temperature of the detected part, the present disclosure removes an interference signal, and realizes direct measurement and tracking of the APPG signal. The present disclosure improves the BP model for the undesired accuracy of the conventional non-invasive BP detection, and provides the transfer learning model for a large calculated amount of the conventional deep learning algorithm. Through model training, the present disclosure realizes non-invasive cardiovascular state screening and BP prediction based on the APPG.

Each embodiment in the description is described in a progressive mode, each embodiment focuses on differences from other embodiments, and references can be made to each other for the same and similar parts between embodiments.

Particular examples are used herein for illustration of principles and implementation modes of the present disclosure. The descriptions of the above embodiments are merely used for assisting in understanding the method of the present disclosure and its core ideas. In addition, those of ordinary skill in the art can make various modifications in terms of particular implementation modes and the scope of application in accordance with the ideas of the present disclosure. In conclusion, the content of the description shall not be construed as limitations to the present disclosure.

What is claimed is:

1. A blood pressure (BP) detection apparatus based on an arteriolar photoplethysmogram (APPG), comprising a ring structure, a photoplethysmogram (PPG) detection unit, a temperature sensing unit, a pressure sensing unit and a microcontroller unit (MCU) provided at a bottom of the ring structure, and spring control units respectively provided at two inner sides of the ring structure, wherein
the PPG detection unit is configured to detect a PPG at a fingertip of a user;
the pressure sensing unit is configured to detect a contact pressure at the fingertip of the user;
the temperature sensing unit is configured to acquire a temperature at the fingertip of the user;
the MCU is connected to the PPG detection unit, the pressure sensing unit and the temperature sensing unit, and configured to calculate an APPG according to the PPG, the pressure and the temperature, and detect a BP according to the APPG; and
the spring control unit is connected to the pressure sensing unit, and configured to adjust the pressure sensing unit according to the pressure.

2. The BP detection apparatus based on an APPG according to claim 1, wherein the PPG detection unit comprises a photoelectric sensor module; and the photoelectric sensor module is configured to detect the PPG at the fingertip of the user, and send the PPG to the MCU through an analog front-end (AFE) or an interface circuit;
the pressure sensing unit comprises a pressure sensor; and the pressure sensor is configured to detect the contact pressure at the fingertip of the user, and send the pressure to the MCU through the AFE or the interface circuit; and
the temperature sensing unit comprises a temperature sensor; and the temperature sensor is configured to acquire the temperature at the fingertip of the user, and send the temperature to the MCU through the AFE or the interface circuit.

3. The BP detection apparatus based on an APPG according to claim 1, wherein the BP detection apparatus further comprises an inertial measurement unit (IMU); and the IMU is connected to the PPG detection unit, the pressure sensing unit, the temperature sensing unit and the MCU, and configured to detect whether data detected by each of the PPG detection unit, the pressure sensing unit and the temperature sensing unit contains interference data, and send interference-removed data to the MCU.

4. The BP detection apparatus based on an APPG according to claim 1, wherein the spring control unit comprises a precision motor, a ratchet structure and a constant-force elastic module; the constant-force elastic module comprises a constant force spring, a movable clamp and a fixed clamp; the precision motor drives the ratchet structure according to the pressure to rotate, thereby adjusting a length of the constant force spring; the movable clamp is sleeved on the constant force spring; the fixed clamp is sleeved on an upper portion of the movable clamp; the constant force spring comprises a top connected to the ratchet structure, and a bottom connected to the bottom of the ring structure; and the fixed clamp and the movable clamp are fixed in the ring structure.

5. The BP detection apparatus based on an APPG according to claim 2, wherein the photoelectric sensor module comprises a plurality of light-emitting diodes (LEDs) and a plurality of photodetectors (PDs); and the plurality of LEDs are centrosymmetric with respect to the plurality of PDs.

6. The BP detection apparatus based on an APPG according to claim 2, wherein the photoelectric sensor module comprises a plurality of LEDs and one PD; and the plurality of LEDs are provided around the PD.

7. The BP detection apparatus based on an APPG according to claim 1, wherein calculating the APPG according to the PPG, the pressure and the temperature, and detecting the BP according to the APPG specifically comprise:
determining a cardiovascular state of the user with a cardiovascular state screening model according to the PPG;
selecting a corresponding BP prediction model from a BP prediction model set according to the cardiovascular state of the user;
calculating the APPG according to the PPG, the pressure and the temperature; and
detecting the BP with the selected BP prediction model according to the APPG.

8. The BP detection apparatus based on an APPG according to claim 7, wherein the cardiovascular state screening model is trained as follows:
constructing a training dataset, the training dataset comprising PPGs and BP data for different cardiovascular states, different genders and different ages, and the cardiovascular states comprising a normal, atrial fibrillation (AF) and coronary arteriosclerosis (CA);
dividing the PPGs into a first set of training data and a second set of training data;
extracting feature information from the first set of training data to obtain multiple types of feature information; and
training a machine learning model through the multiple types of feature information and the cardiovascular states corresponding to the PPGs to obtain the cardiovascular state screening model.

9. The BP detection apparatus based on an APPG according to claim 8, wherein the BP prediction model set is trained as follows:
training multiple deep learning models through the second set of training set in the different cardiovascular states and the corresponding BP data to obtain the BP prediction model set, the BP prediction model set comprising multiple BP detection models, and the BP detection models being configured to respectively detect BPs in the cardiovascular states.

10. The BP detection apparatus based on an APPG according to claim 7, wherein calculating the APPG according to the PPG, the pressure and the temperature specifically comprises:
determining an adjustment amount of the spring control unit according to the pressure;
acquiring the PPG when the LEDs have a wavelength of λ₁ and a wavelength of λ₂ under the adjustment amount of the spring control unit;
determining a scale factor according to the PPG;
determining a revised item of the scale factor according to the temperature; and
calculating the APPG according to the PPG, the scale factor and the revised item of the scale factor.
